(19) United States
(12) Patent Application Publication
(10) Pub. No.: US 2024/0247323 A1
(43) Pub. Date: Jul. 25, 2024

(54) MULTIPLEXED PCR ASSAY WITH POINT OF CARE SAMPLE

(71) Applicant: ChromaCode, Inc., Carlsbad, CA (US)

(72) Inventors: Aditya RAJAGOPAL, Carlsbad, CA (US); Alex DICKINSON, Carlsbad, CA (US); Vernon NORVIEL, Carlsbad, CA (US); Lucien JACKY, Carlsbad, CA (US); John ANNALORO, Carlsbad, CA (US); John ALVARADO, Carlsbad, CA (US)

(21) Appl. No.: 17/918,802

(22) PCT Filed: Apr. 13, 2021
(86) PCT No.: PCT/US2021/027127

§ 371 Date: Oct. 13, 2022

Related U.S. Application Data
(60) Provisional application No. 63/024,965, filed on May 14, 2020.
(60) Provisional application No. 63/009,994, filed on Apr. 14, 2020.

(51) Int. Cl. C12Q 1/70; C12Q 1/6811; C12Q 1/6844

(52) U.S. Cl. CPC C12Q 1/701; C12Q 1/6811; C12Q 1/6844; C12Q 2600/16

(57) ABSTRACT

Methods, systems, compositions and kits for using a multiplexed assay on a sample that is obtained from a subject are disclosed. The methods, systems, compositions and kits may comprise obtaining a sample from a subject that is in a location distal from where the assay is performed. The subject may obtain the sample without the presence of another individual. The methods, systems, compositions and kits may allow an assay to be run without addition of a reagent after the sample is received from the subject and prior to running an assay.

### CROSS REFERENCE

This application is a U.S. National Stage application of International Application No. PCT/US2021/027127, filed Apr. 13, 2021, which claims the benefit of U.S. Provisional Application No. 63/009,994, filed Apr. 14, 2020, and U.S. Provisional Application No. 63/024,965, filed May 14, 2020, which applications are incorporated herein by reference in their entirety.

### SEQUENCE LISTING

The instant application contains a Sequence Listing which has been submitted electronically in ASCII format and is hereby incorporated by reference in its entirety. Said ASCII copy, created Jan. 22, 2024, is named 44158-722_831_SL25.txt and is 8,713 bytes in size.

### BACKGROUND OF THE INVENTION

Detection of nucleic acid sequences are used for a wide variety of purposes. Detection of a particular nucleic acid sequence in a gene of a subject may indicate that the subject may have a particular disorder or be more prone to having a particular disorder. Detection of nucleic acid sequences may be used to detect an infection by detecting a gene or nucleic acid sequence of a pathogen. PCR may be used to amplify nucleic acids for analysis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. An understanding of the features and advantages of the present invention will be obtained by reference to the following detailed description that sets forth illustrative embodiments, in which the principles of the invention are utilized, and the accompanying drawings of which:

FIG. 1 illustrates a schematic of the assay as viewed from a subject that is providing a sample.

FIG. 2 illustrates a schematic of the steps of the assay once the sample is received from a subject.

FIG. 3 illustrates a schematic of the interface of the assay results and downstream individuals who may use the assay results to guide decisions.

FIG. 4 illustrates a general schematic of the assay steps and results.

FIG. 5 illustrates a schematic regarding the potential information that can be gathered to quantify a patient's or subject's risk and subsequent diagnosis or treatment recommendation.

FIGS. 6A-6B shows graphs obtained from an assay on target nucleic acids and assays on different dilutions of the sample.

FIG. 7 shows graphs obtained from an assay on target nucleic acids assays on different dilutions of the sample.

FIG. 8 shows graphs obtained from an assay on target nucleic acids assays on different dilutions of the sample and detection performed in multiple channels.

FIG. 9 shows graphs obtained from amplification of a nucleic acid from dilutions of a saliva sample.

FIG. 10 shows a schematic of the configuration of the assay for multiplexing and detecting different target nucleic acids.

FIG. 11A-B show graphs of data corresponding to the detection of multiple target nucleic acids in the assay in a sample.

FIG. 12 shows a schematic for designing primers for the assay.

FIG. 13 shows graphs of an assay using designed primers for amplification of target nucleic acids.

FIG. 14 shows graphs of the results of an assay run using saliva samples containing SARS-CoV-2 viral RNA at different reverse transcription temperatures.

FIG. 15A-B shows graphs of the results of an assay run using saliva samples containing SARS-CoV-2 viral RNA under different preheating and annealing conditions.

FIG. 16 shows graphs of the results of an assay run using saliva samples containing SARS-CoV-2 viral RNA in different buffer conditions.

### DETAILED DESCRIPTION OF THE INVENTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “channel,” “color channel,” or “optical channel”, as used herein, generally refers to a range of wavelengths. The channel may be set or determined based on particular filters which remove or filter out particular wavelengths. The terms “channel,” “color channel,” and “optical channel” can be used interchangeably.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles (Saiki et al 1988).

Real-Time PCR (qPCR) is a process of monitoring a PCR reaction by recording the fluorescence generated either by an intercalating dye such as SYBR Green or a target-specific reporter probe at each cycle. This is generally performed on a Real-Time PCR instrument that executes thermal cycling of the sample to complete the PCR cycles and at a specified point in each cycle measures the fluorescence of the sample in each channel through a series of excitation/emission filter sets.

Primers, or “amplification oligomers,” used herein interchangeably, refer to an oligonucleotide or nucleic acid configured to bind to another nucleic acid and facilitate one or more reactions, for example, transcription, nucleic acid synthesis, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid or may bind to the non-coding strand of a nucleic acid. A reverse primer may bind to the coding strand of a nucleic acid or may bind to the non-coding strand of a nucleic acid.

Frequently, the target-specific oligonucleotide probe is a short oligonucleotide complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR (Holland et al. 1991). The TaqMan oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

Provided herein are methods for detection of nucleic acids sequences from a subject that may indicate a disease state, disorder, or the presence of a pathogen, wherein the subject does not need to be near the location in which the assay for the nucleic acids is performed. Current procedures to test for nucleic acids in a subject may require a subject to physically be present at a testing facility to be tested. For example, the subject may be required to go to a hospital in order to be tested for a disease. The subject may need to provide a sample directly to the testing facility in order to minimize any loss of stability in the sample and to improve the integrity of a test. Additionally, a sample may need to be provided to the test facility in a larger quantity in order for detection of a nucleic acid occur. The larger quantity may necessitate that the sample be obtained by a trained individual who is not the subject.

However, in multiple cases, it may be impractical or difficult for the subject to be at a particular physical location, or with a particular trained individual, in order to properly be tested. There remains a need to obtain samples from a subject that provide adequate sample material to be tested, without the subject present at a particular location, or without the aid of another individual.

The present disclosure provides methods, kits, compositions for a subject to provide an adequate sample for testing. The sample provided from the subject may become degraded or experience a loss of integrity if it is not prepared in a manner to preserve the integrity of the sample. In the case of a sample which is obtained at a location distal to testing facility, the sample may degrade in the time between when the sample was obtained by the subject and when the sample arrives at the testing facility. The methods and compositions herein may allow the maintenance of sample integrity throughout the time the sample is transported to the testing facility. The sample integrity may be maintained throughout the assay and provide sufficient amounts of material for the assay to be performed and produce an accurate result.

The present disclosure provides methods, kits, compositions for a subject to provide an adequate sample for testing without the addition of a reagent prior performing an assay once the sample has been obtained. The sample provided from the subject may be diluted prior to the assay. The sample may not undergo extraction or purification of nucleic acids prior to being assayed, once the sample has been received. The sample integrity may be maintained throughout the assay and provide sufficient amounts of material for the assay to be performed and produce an accurate result.

In various aspects described herein the sample collected is not subjected to processing before assaying after the collection of the sample. The sample may not be chemically processed prior to the assaying. The sample may not be amplified prior to the assay reaction. The sample may not be enriched prior to performing the assay reaction. The sample may not be subjected to a purification reaction prior to performing the assay reaction.

In various aspects described herein a sample is a collected from a subject. The biological sample may be collected using a sample collection tube or vessel. The biological sample may be collected using a sample collection tool. The sample collection tool may comprise swab. The sample may be collected by the subject without the help of another individual. The sample may be collected in a subject's home away from a medical facility. The sample may be collected based on a set of instructions provided in a kit to the subject.

In various aspects described herein, a subject may collect a sample which is sent to another location for an assay to be performed. The sample may be mailed to a testing facility. The sample may be mailed to a central processing facility which may categorize sample or encode the samples such that personal information is encoded and not immediately available to an individual performing the assay. For example, the sample may be identified by an alphanumeric identifier, for example, to allow for processing of the sample or analysis of results without the subject's personal information.

In various aspects described herein, the results of the assay may be outputted as a report. The report may be outputted to a remote computer database. The report may be outputted such that it is accessible to the subject. The report may be outputted such that it is accessible to a medical provider or to an institution. The report may be accessible via a smart phone. The report may be accessible via an application. The report may be used in the conjunction with a subject medical record. The report may comprise medical recommendations.

The sample collection tube may comprise an additional reagent. For example, the sample collection tube may comprise a reagent for maintaining the integrity of nucleic acids in the sample. For example, the sample collection tube may comprise a reagent for slowing or preventing the degradation of nucleic acids in the sample. The additional reagent may be a reducing agent. The additional reagent may be a chelator. For example, the chelator may be ethylenediaminetetraacetic acid (EDTA). The additional reagent may be a salt, a buffer or buffer salt. For example, the additional reagent may be Tris (2-amino-2-hydroxymethyl-propane-1,3-diol). The additional reagent may be sodium acetate. The additional reagent may be an alcohol. For example, the reagent may be ethanol. In another example, the sample collection tube may comprise a reagent that indicates if a sample is present. The sample collection tube may comprise a reagent that indicate that a sample is compromised or otherwise unusable for an assay.

The sample may undergo heating steps prior to assaying for nucleic acids. The heating step may deactivate or attenuate a pathogen or pathogenic component. The heating may allow the sample to be less infectious thereby allowing the assay to be performed with less risk to the individual performing the assay. The sample may be heated for a period of time. For example, the sample may be heated for at least 1, 2, 3, 4, 5, 6, 7, 8, 9, 10, 11, 12, 13, 14, 15, 16, 17, 18, 19, 20, 25, 30, 35, 40, 45, 50, 60, or more seconds. the sample may be heated for no more than 1, 2, 3, 4, 5, 6, 7, 8, 9, 10, 11, 12, 13, 14, 15, 16, 17, 18, 19, 20, 25, 30, 35, 40, 45, 50, 60, or less seconds. The sample may be heated for at least 1, 2, 3, 4, 5, 6, 7, 8, 9, 10, 11, 12, 13, 14, 15, 16, 17, 18, 19, 20, 25, 30, 35, 40, 45, 50, 60, or more minutes. The sample may be heated for at no more than 1, 2, 3, 4, 5, 6, 7, 8, 9, 10, 11, 12, 13, 14, 15, 16, 17, 18, 19, 20, 25, 30, 35, 40, 45, 50, 60, or less minutes. The heating may be performed in pulses such that the sample is subjected to heating for a period of time and then taken away from the heat source for a period of time.

In various aspects disclosed herein, the results of the assay may be used to determine that a gene or infectious pathogen is present in an individual. The assay may be used to generate a genotype or determine the presence of an infectious pathogen in a subject. The assay results may be used to calculate a risk based on the presence of genes or infectious agents in a sample. For example, the risk may be calculated based on the presence of a particular SNP in a gene or a particular pathogen.

In various aspects disclosed herein, the results of the assay may be used to generate a risk score or probability of risk. Using the results of assay, for example, the genotype of an individual or the presence of a genetic marker may indicate that a subject is at a higher risk for infection, or a higher risk of complications or mortality due to an infection. The markers may be related to immune system response or a related to proteins involved in or targeted by viruses for entry or other pathways for infection. The results of the assay may indicate that a pathogen is absent or present. The results may indicate that a particular strain or serotype is present. The results may be combined with other data relating to the subject medical history.

Methods as described herein may be used to quantify 1, 2, 3, 4, 5, 6, 7, 8, 9, 10, or more analytes in a sample volume. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The mixture may further comprise other reagents (e.g., amplification reagents) including, for example, oligonucleotide primers, dNTPs, a nucleic acid enzyme (e.g., a polymerase), and salts (e.g., Ca2+, Mg2+, etc.). Next, the mixture may be used in a quantitative Polymerase Chain Reaction (qPCR), whereby a plurality of signals may be generated. At least one of the plurality of signals may be detectable in multiple color channels. Based on the detecting, the nucleic acid target in the sample may be quantified. A signal of the plurality of the signal may be detectable in only one color channel. For example, a first signal of the plurality of signals is detected in multiple color channels, and a second signal is detectable in only one color channel, and the analytes correlated to the first and second signals may be quantified. In another example, a first signal of the plurality of the signals is detected in a first two color channels and a second signal of the plurality of signals is detected in a second two color channels, and at least one of the channels in the first two color channels and the second two color channels is the same or substantially the same color channel. A signal may be detected or measured at least 1, 2, 3, 4, 5, 6, 7, 8, 9, 10, 11, 12, 13, 14, 15, 20, 30, 40, 50, or more channels. A signal may be detected or measured in no more than 1, 2, 3, 4, 5, 6, 7, 8, 9, 10, 11, 12, 13, 14, 15, 20, 30, 40, 50, or less channels.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In multiple aspects as described herein, signals and data relating to the detection of the signals are subjected to processing in order for the signals and data to be used for subsequent steps or downstream methods. The processing may use mathematical algorithms to analyze or process the signal data. In some case, the processing may use data obtained from the instrument or detector. The processing may use data obtained from multiple channels, or a single channel. In some cases, the processing may use data from channels that are not expected to correlate with a signal from a given probe or fluorophore. For example, the data may include data obtained from a reference channel in which a background signal is obtained. The processing may use data obtained from all available channels of a given detection device.

The mathematical algorithms used for data processing may include expectation maximization, nearest neighbor analysis, basic model parameterization, Bayesian estimation, or combinations thereof. The mathematical algorithm may use a process parameter. Examples or process parameters include parameters for threshold cycles, amplitudes, or slopes.

The processing of data may comprise plotting the data. Processing the data may use plotting functions to analyze individual or multiple points such to calculate a correlation or to better visualize data. The data may be plotted as a curve. The data may be represented as a kinetic signature, wherein the signal amplitude may plotted be against a metric of time (such as cycles or seconds) or a metric that can be mathematically transformed into a metric of time (such as a frequency). The data may be fit to a variety of functions in order to derive parameters from the data. For example, a plotted data may be fit to a linear function such that a slope parameter can be derived from the data.

Processing of the data may also comprise identifying a data point as belonging to a data set. In some cases, multiple analytes are analyzed simultaneously, wherein the signal generated from analytes may comprise overlapping signals from different analytes. Processing the data may alleviate the overlapping signals or may correlate the data points to different data set in which the signal is detected via another method or alternative channel or detector.

In various aspects, reference conditions are used for comparing with data sets or for deriving reference parameters such as reference quantification parameters. Reference conditions may comprise a known concentration of a reagent or analytes. Reference conditions may comprise a known reaction condition such as the temperature or pH of a solution. For example, the reference condition may comprise a concentration, amount, or quantity of a reference nucleic acid. The reference condition with the known parameter may be used to extrapolate, interpolate or otherwise calculate a concentration, quantity, or amount of another nucleic acid in a separate sample. Reference conditions may comprise signals which may be detected or processed or as described elsewhere herein for any other signal. For example, data from the reference condition may be used to generate reference data which in turn may be parameterized by mathematical algorithms to generate reference quantification parameters. The generation of reference quantification parameters can be used to directly compare to generated quantification parameters of a data set or can be used to calculate a quantification parameter based on for example, parameterization, fitting, extrapolation, interpolation, or estimation of the data set or a parameter of the data set.

References conditions may be specific to a type of reaction. Reference conditions may comprise conditions for an amplification reaction. Examples of amplification reactions are described elsewhere herein. Reference conditions may, for example, comprise a concentration of a polymerase or a type of polymerase. For example, reference conditions may comprise a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

In various aspects, nucleic acid molecules may be quantified. The quantification may be an absolute quantification. For example, the molarity of a starting amount of a nucleic acid may be determined. This may be determined using a reference condition or amount with a known molarity of nucleic acid. The quantification may be a relative quantification. For example, a second nucleic acid may be determined to have a larger starting amount than a first nucleic acid.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.). A nucleic acid target may be a nucleic acid from a pathogen (e.g., virus, bacteria, etc.). A nucleic acid target may be a nucleic acid suspected of comprising one or more mutations.

In some embodiments, the present disclosure provides a multiplexed assay for simultaneous amplification, detection, and or/quantification of at least one analyte in a sample. In some embodiments the methods of the disclosure may be used to detect and/or quantify at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, 2000, 3000, or more different target analytes in a sample. In some embodiments the methods of the disclosure may be used to detect and/or quantify at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, 2000, 3000, or less different target analytes a sample.

In some cases, assays may be run using the reagents in the chemical composition. Assay may use a reagent to perform a reaction. The reaction may comprise a hybridization reaction. For example, the reagent may comprise a nucleic acid and hybridize with another nucleic acid. The nucleic acid and the another nucleic acid may be complementary to one another. The reaction may comprise an extension reaction. For example, the reaction may comprise extending a nucleic molecule by the addition of a nucleotide. The reaction may comprise a polymerase chain reaction.

Methods as described herein may be performed without the use of immobilization, separation, mass spectrometry, or melting curve analysis. For example, the sample reagents and analytes may all be in solution. The analytes may be analyzed without needing to purify or physically separate the analytes from one another. Identification of the analytes may be performed without obtaining a mass of the analytes via mass spectrometry or any similar technique. Additionally, the methods may be used without observing a melting reaction and plotting the signal against a temperature. For example, an analyte may be identified without subjecting the analyte to temperature gradient in order to analyze a specific temperature in which an analyte goes through a physical or chemical change. The methods as described herein may be corroborated via techniques using immobilization, separation, mass spectrometry, or melting curve analysis. For example, the melting curve may be used to verify a number of different amplicons or detecting a presence of an amplicon.

Any number of nucleic acid targets may be detected using assays of the present disclosure. In some cases, an assay may unambiguously detect at least 3, 4, 5, 6, 7, 8, 9, 10, 11, 12, 13, 14, 15, 20, 30, 40, 50 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a at least a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in at least two channels, and a second reaction may be performed in a second sample solution volume and generate a fluorescent signal in the same color channel or a different color channel, thereby generating two measurements for comparison.

Each oligonucleotide probe may be labeled with a fluorophore. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule.

The fluorescent molecules may have a distinct or known signature of excitation or emission wavelength of electromagnetic radiation. The detection of a fluorescent molecule signature may comprise identifying an amplitude or amplitudes of signal at different wavelengths. In some cases, the fluorescent molecule signature may comprise a signal at wavelengths that overlaps with wavelengths that may be generated by reagents in the chemical composition. In some cases, the excitation wavelength of the molecule may comprise a signal that overlaps with wavelengths that may be generated by reagents in the chemical composition. In some cases, the signals of the reaction and the fluorescent molecule may be simultaneously detected. Non-limiting examples of fluorescent molecules that may be used include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof.

### Amplification

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Nucleic Acid Targets

A nucleic acid target of the present disclosure may be derived from a biological sample. A biological sample may be a sample derived from a subject. A biological sample may comprise any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be, for example, blood, plasma, serum, urine, saliva, mucosal excretions, sputum, stool or tears.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene. The nucleic acid target may be a nucleic acid derived from an infectious agent. For example, the nucleic acid target may comprise a sequence of an influenza gene. The influenza gene may be an influenza A PB gene, an influenza HA gene, or an influenza B NS gene. For example, the nucleic acid target may comprise a sequence of a SARS-CoV-2 gene. The nucleic acid target may comprise a sequence that is indicative of the presence of the flu or COVID-19 in a subject. The SARS-CoV-2 gene may be a SARS-CoV-2 N gene or a SARS-CoV-2 N gene. The SARS-CoV-2 N gene may be a N1, N2 or N3 gene. The nucleic acid target may comprise a sequence indicative of a serotype. For example, the nucleic acid target may be an influenza HA gene sequence corresponding to the H1 or H3 serotype. The nucleic acid target may comprise a single nucleotide polymorphism (SNP). The nucleic acid target may allow a genotype to be determined. The nucleic acid target may be a region of the human genome that indicates a predisposition for a particular disease. For example, a particular mutation or SNP of in a subject may be associated with an increased risk of infection of a particular pathogen. For example, the detection of both a pathogenic nucleic acid sequence and the presence of the mutation in the subject's genome may indicate the subject is at a high risk.

Markers for a predisposition for a particular disease may be detected by assays as disclosed elsewhere herein. The markers for predisposition may be related to other existing conditions, for example, another disease such as diabetes, cancer, heart disease, or a condition that results in the individual being immune compromised. The markers may be related to immune response, for example, markers that indicate an intensity of a response to an antigen. The markers may be related to specific mutations or SNPs that are associated with higher infections. For example, a SNP of a receptor may have a higher affinity to a virus, thereby allowing the virus to more easily recognize and infect a cell.

Nucleic acid targets may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample may undergo an extraction to extract molecules used in the assay. For example, the extraction may use a column to bind or interact with a molecule. For example, an RNA extraction kit may be used such as a Qiagen RNA mini kit to extract or isolate RNA. A sample may be not processed after being added to a sample collection tube. A sample may not undergo any processing that purifies or enriches for nucleic acids and the assay may be performed directly on the sample. A sample may not require processing to purify or enrich for nucleic acids. The assay may have a sensitivity such that a sample that does not undergo processing may be assayed and result in an accurate result. A sample may not undergo an extraction reaction such to extract nucleic acids after the sample is received and prior to running an assay. A sample may not undergo an extraction reaction such to purify nucleic acids after the sample is received and prior to running an assay. A sample may be diluted. A sample may be diluted at least at 1:1, 1:2, 1:3, 1:4, 1:5, 1:6, 1:7, 1:8, 1:9, 1:10, 1:11, 1:12, 1:13, 1:14, 1:15:1:16:1:17, 1:18, 1:19, 1:20, 1:30, 1:40, 1:50, 1:60, 1:70, 1:80, 1:90, 1:100, or 1:1000, 1:10000, 1:100000 or more. A sample may be diluted at no more than at 1:1, 1:2, 1:3, 1:4, 1:5, 1:6, 1:7, 1:8, 1:9, 1:10, 1:11, 1:12, 1:13, 1:14, 1:15:1:16:1:17, 1:18, 1:19, 1:20, 1:30, 1:40, 1:50, 1:60, 1:70, 1:80, 1:90, 1:100, or 1:1000, 1:0000, 1:100000, or less. The sample may be diluted in a buffer or a solution. For example, the sample may be diluted in Tris-Ethylenediaminetetraacetic acid (TE) buffer. The sample may be diluted with a solution comprising alcohol. The sample may be diluted with a solution comprising sodium acetate.

The assays, methods, and systems of the present disclosure may be able to detect a nucleic acid target in a sample without sample processing steps. For example, the detection may be performed on a sample that has not been purified or enriched for a nucleic acid. An assay may take a sample comprising nucleic acids from a subject, subject the nucleic acids to a reverse transcriptase and polymerase chain reaction directly, without a step or purification or enrichment, and detect the nucleic acid target. An assay may, for example, dilute a sample comprising nucleic acids from a subject into a suitable buffer, subject the nucleic acids to a reverse transcriptase and polymerase chain reaction directly, without a step or purification or enrichment, and detect the nucleic acid target.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

In various aspects disclosed elsewhere herein, reactions are performed. A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

### Oligonucleotide Primers

In various aspects disclosed elsewhere herein, oligonucleotide primers are used. An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences. For example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

A set of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, the primer may be configured to amplify sequences derived from influenza virus, coronavirus, respiratory syncytial virus, hepatitis virus, herpesvirus, or papillomavirus. The primer may be configured to amplify a SARS-CoV-2 N gene. The primer may be configured to amplify a SARS-CoV-2 E gene. The primer may be configured to amplify an influenza A PB1 gene, influenza A Hi gene, influenza B NS gene. The primer may comprise a sequence in Table 1. The primer may comprise a sequence with at least a 50%, 55%, 60% , 65% , 70% , 75%, 80% , 85% , 90% , 95%, 99% or more homology with a sequence in Table 1.

TABLE 1

| SEQ ID: | Primer Name | Primer Sequence |
|---|---|---|
| 1 | 2019-nCoV_N1-F1 | GACCCCAAAATCAGCGAAAT |
| 2 | 2019-nCoV_N1-R1 | TCTGGTTACTGCCAGTTGAATCTG |
| 3 | 2019-nCoV_N2-F1 | TTACAAACATTGGCCGCAAA |
| 4 | 2019-nCoV_N2-R1 | GCGCGACATTCCGAAGAA |
| 5 | hRNase P Extraction Control F1 | AGATTTGGACCTGCGAGCG |
| 6 | hRNase P Extraction Control R1 | GAGCGGCTGTCTCCACAAGT |
| 7 | InfA_PB1_dom_F1 | CTCAGACGATTTTGCCCTCATAGT |
| 8 | InfA_PB1_dom_F2 | GAATACAAGCAGGAGTGGATAGATTCT |
| 9 | InfA_PB1_dom_R1 | CACTAACTTGCAGGTCCTATAGAATCT |
| 10 | InfA_PB1_dom_R2 | GCTAGTGAATTCAAATGTCCCTGTT |
| 11 | InfAH1_dom_F1 | CGGCTGCTTTGAATTTTACCACAA |
| 12 | InfAH1_dom_F2 | GTTGATGATGGTTTCCTGGACAT |
| 13 | InfAH1_dom_R1 | GCTTCCTCTGAGTATTTTGGGTAGT |
| 14 | InfAH1_dom_R2 | CGTGATAGTCCAAAGTTCTTTCGTT |
| 15 | InfAH3_dom_F1 | GGAAGAGGACAAGCAGCAGATC |
| 16 | InfAH3_dom_F2 | GCTTCTTGTTGCCCTGGAGAA |
| 17 | InfAH3_dom_R1 | CAATCTGATGGAATTTCTCGTTGGT |
| 18 | InfAH3_dom_R2 | TGCAGGCATTGTCACATTTGT |
| 19 | InfB_dom_F1 | GATGATCTTACAGTGGAGGATGAAGA |
| 20 | InfB_dom_R1 | CTTCTGGTGATAATCGGTGCTCTT |
| 21 | RSVA_dom_F1 | TGGTACTGTGACAATGCAGGATCAGT |
| 22 | RSVA_dom_R1 | AGCAAGAAGAGGAAACGAAGATTTCTGGG |
| 23 | RSVB_dom_F1 | GCATATGTTGTACAGCTACCTATCTATGG |
| 24 | RSVB_dom_R1 | CCATCCTCTATCAGTCCTTGTTAAACA |

In some aspects, a mixture may include one or more synthetic (or otherwise generated to be different from the target of interest) primers for PCR reactions.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule.

In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

In various aspects disclosed elsewhere herein, oligonucleotide probes are used. Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes may be used. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may comprise a signal tag with about equal emission wavelengths. In some cases, each oligonucleotide probe comprises an identical fluorophore. In some cases, each oligonucleotide probe comprises a different fluorophore. In some case, each fluorophore is capable of being detected in a single optical channel. In other case, a fluorophore may be detected in multiple channels. In some cases, an oligonucleotide probe may have similar or the same detectable agent or fluorophore as another oligonucleotide probe in the sample. In some cases, an oligonucleotide probe may have a different detectable agent or fluorophore as compared to another oligonucleotide probe in the sample. In some cases, an oligonucleotide probe may have similar sequence or be capable or binding a similar sequence as another oligonucleotide probe in the sample. In some cases, an oligonucleotide probe may have a different sequence or be capable of binding a different sequence as compared to another oligonucleotide probe in the sample.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions. etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

In some aspects, the probe may be configured to hybridize, anneal or be homologous to sequences derived from influenza virus, coronavirus, respiratory syncytial virus, hepatitis virus, herpesvirus, or papillomavirus. The probe may be configured to hybridize, anneal or be homologous to sequences of a SARS-CoV-2 N gene. The probe may be configured to amplify a SARS-CoV-2 E gene. The probe may be configured to hybridize, anneal or be homologous to sequences of an influenza A PB1 gene, influenza A H1 gene, influenza B NS gene. The probe may comprise a sequence in Table 2. The probe may comprise a sequence with at least a 50%, 55%, 60%, 65%, 70%, 75%, 80%, 85%, 90%, 95%, 99% or more homology with a sequence in Table 2.

TABLE 2

| SEQ ID: | Probe Name | Probe Sequence |
|---|---|---|
| 25 | 2019-nCoV_N1-P | ACCCCGCATTACGTTTGGTGGACC |
| 26 | 2019-nCoV_N2-P | ACAATTTGCCCCCAGCGCTTCAG |
| 27 | hRNase P cdc P1 | TTCTGACCTGAAGGCTCTGCGCG |
| 28 | InfA_PB1_dom_P1 | TGAGGGAATACAAGCAGGAGTGGA |
| 29 | InfA_PB1_dom_P2 | AGGACCTGCAAGTTAGTGGGAATCA |
| 30 | InfAH1_dom_P1 | TGCATGGAAAGTGTCAAGAATGGGA |
| 31 | InfAH1_dom_P2 | TGGACTTACAATGCCGAACTGTTGGT |
| 32 | InfAH3_dom_P1 | aGCACTCAAGCAGCAATCGATCAAA |
| 33 | InfAH3_dom_P2 | aGCAACTGAGGGAAAATGCTGAGGA |
| 34 | InfB_dom_P1 | aGGACATTCAAAGCCAATTCGAGCAGC |
| 35 | RSVA_dom_P1 | GTTAGGTGTTGGATCTGCAATCG |
| 36 | RSVB_dom_P1 | CAGCCTTGTTTGTGGATAGTAGAG |

A probe may be provided at a specific concentration. In some cases, a second nucleic acid probe is provided at a concentration of at least about 2×, about 3×, about 4×, about 5×, about 6×, about 7×, about 8×, or more. In some cases, a second nucleic acid probe is provided at a concentration of at most about 8×, about 7×, about 6×, about 5×, about 4×, about 3×, or about 2×. In some cases, a second nucleic acid probe is provided at a concentration of about 2×, about 3×, about 4×, about 5×, about 6×, about 7×, or about 8×. X may be a concentration of a nucleic acid probe provided in the disclosed methods. In some cases, X is at least 50 nM, 100 nM, 150 nM, 200 nM, 250 nM, 300 nM, 350 nM, 400 nM, 450 nM, 500 nM, or greater. In some cases, X is at most 500 nM, 450 nM, 400 nM, 350 nM, 300 nM, 250 nM, 200 nM, 150 nM, 100 nM, or 50 nM. X may be any concentration of a nucleic acid probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

An oligonucleotide probe may comprise a detectable label. A detectable label may be a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. The number of signals generated in an assay may correspond to the number of oligonucleotide probes and nucleic acid targets present.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a signal intensity threshold. A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules. The presence of a plurality of signals may be correlated with a combination of targets. The presence of a plurality of signals may be correlated with a unique combination of targets. For example, the detection of a particular plurality of signals may indicate the presence or absence of a unique or particular combination of targets.

### Kits

The present disclosure provides kits for sample collection. The kit may comprise a sample collection vessel or sample collection tube. The kit may comprise a sample collection tool or an object that can obtain a sample via the contact of cells or nucleic acids from the subject and transfer sample to a sample collection vessel or tube. The sample collection tool may comprise a swab.

The present disclosure also provides kits for performing assays or analysis of assay results. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers (or “amplification oligomers”) as described herein. A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe. Kits may comprise instructions for using any of the foregoing in the methods described herein.

Kits provided herein may be useful in, for example, calculating at least first and second sums, each being a sum of multiple target signals corresponding with a first and second target nucleic acid.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution monitoring the spread of a disease. The results may also be sent to the subject directly. The subject, medical provider, or other institution may be able to access the remote server such review or analyze the results. For example, the results may then be transmitted to another institution/or medical professional for monitoring or for providing recommendations for the subject. In additional to the data generated for the detection of targets, the data may be used to monitor a geographical location of the assay or subject, for example to allow monitoring of the transmission of a disease. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor” and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Assay for Viral RNA on Sample Mailed by Subject

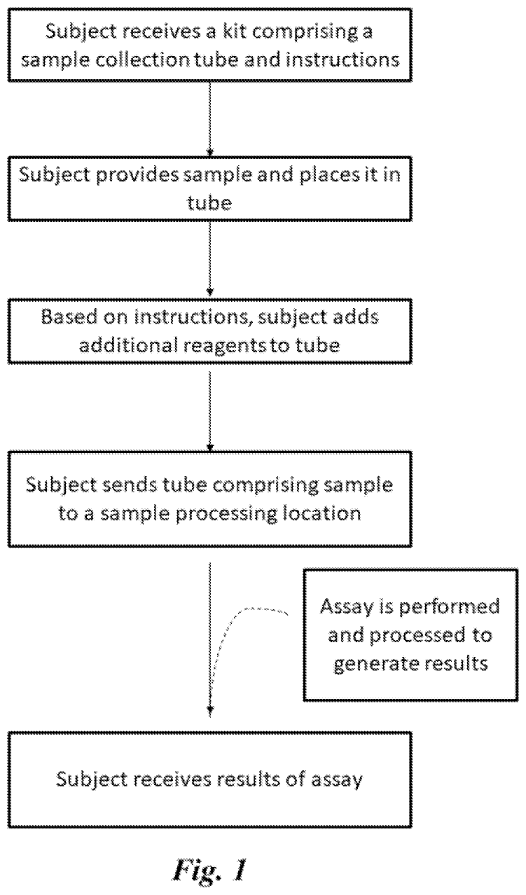

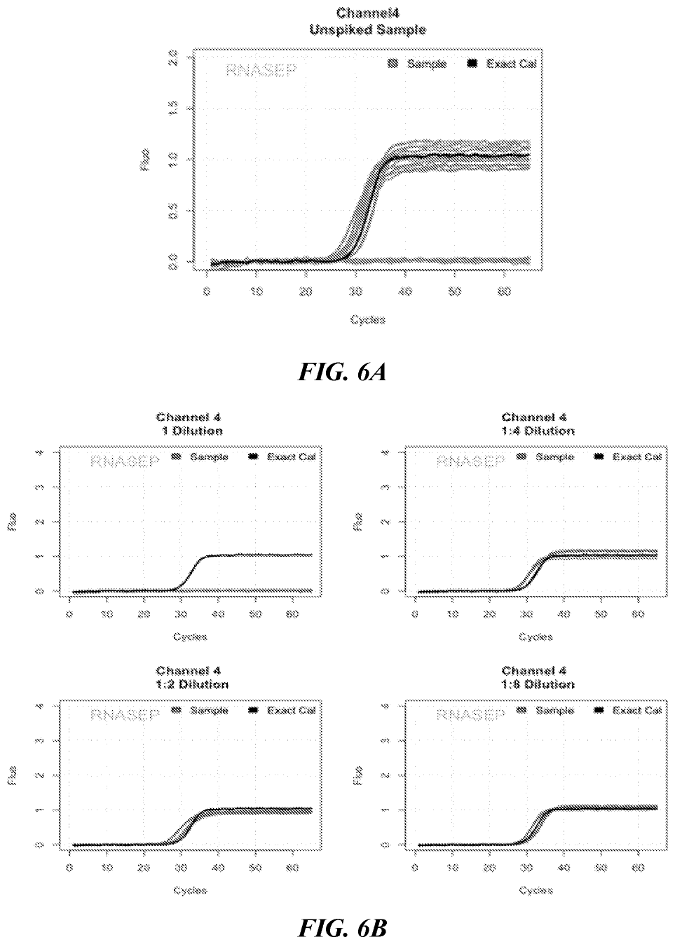

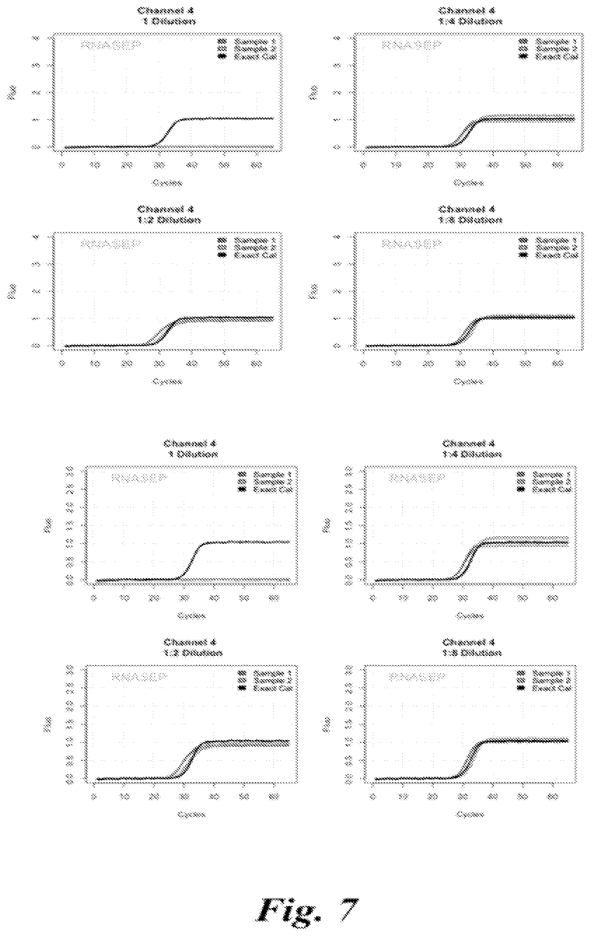

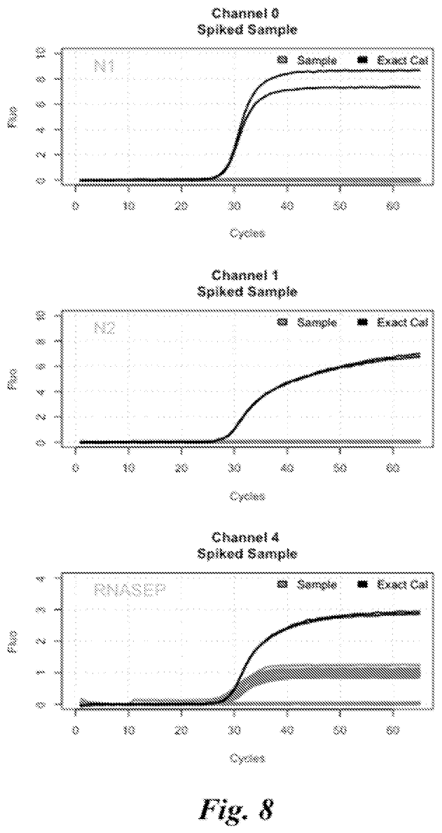

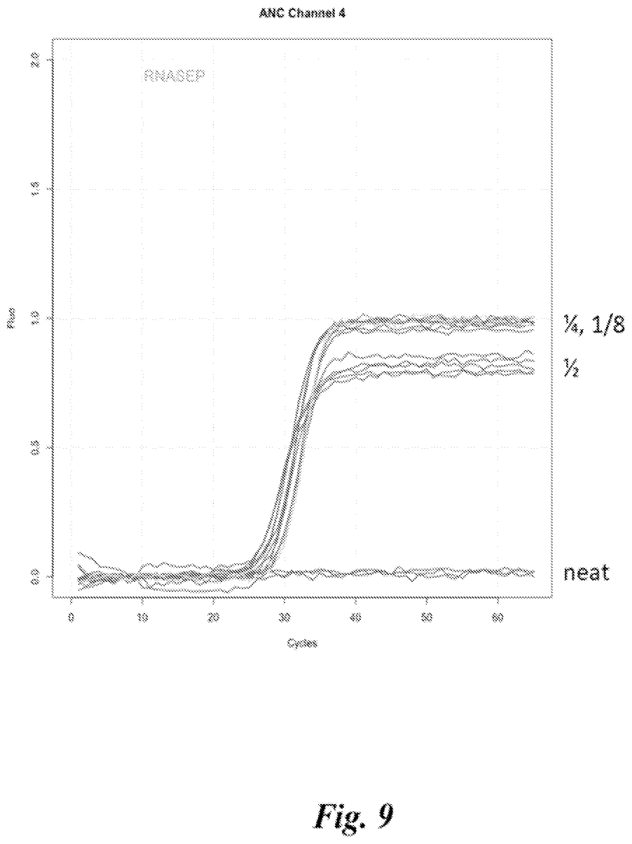

The subject or subjects is suspected of having COVID-19. The subject provides a saliva sample by using swab or other collection tube to obtain a sample and places the sample in a sample tube of the type provided by Ancestry.com. The subject may optionally be provided with a tube via a mailing service or other delivery system which may allow the subject to obtain a tube and then provide a sample away from other individuals. The subject(s) may be a man or a woman. The tube may also be accompanied by an instruction pamphlet or other documentation to provide information to the subject about how to provide a sample (e.g. how to take the sample, how to return the sample tube). FIG. 1 shows a schematic of the steps of providing a sample as done by the subject. The tube is sent to a testing location which receives the saliva sample. The saliva sample is subjected to heat to attenuate or inactivate potential viral particles and may help render the sample to be less infectious. The saliva sample is then subjected to a dilution and is split into multiplets the allow multiple test. The sample may also be subjected to multiple different dilution parameters to improve the quality of the assay results. The dilution may allow component that may interfere with the assay to be diluted to concentrations that are cause less of an interference while maintain sufficient amounts of material to assay. Additionally, no additional extraction of nucleic acids or purification of the nucleic acids from the rest of the sample is performed. A PCR based assay which comprises primers and probes specific to a target nucleic acid is run by combining the primers, probes and enzymes with the sample dilutions. The sample is then thermocycled and the detection of signals from the probes is performed. FIGS. 6-9 show example data performed to detect a target nucleic acid in an unspiked and spiked sample. FIG. 6A and FIG. 9 shows the fluorescence of the sample under multiple dilutions when detecting for RNase P. The fluorescence of the sample indicates that the target nucleic acids is present. FIG. 6B shows the assay performed on multiple dilutions of the sample. The “1 dilution” which is an undiluted sample does not show a signal as the line corresponding to “sample” does not increase in fluorescence. The 1:2 dilution, 1:4 dilution, 1:8 dilution shows an increase in signal and is able to detect the RNase P. FIG. 9 shows these dilutions detected in the same channel, with the ½, ¼, and ⅛ dilutions showing an amplification signal, whereas the neat (undiluted) sample does not show amplification and a signal. FIG. 7 shows additional results from multiple runs of the assay, all demonstrating the detection ins RNase P in the diluted samples, with no signal in the undiluted sample. FIG. 8 shows a multiplexed assay detecting presences or absences of N1, N2 and RNase P in different channels using dilutions of a sample, wherein each dilution was spiked with 250 copies/ul of Exact Science RNA. As with the unspiked samples, the dilutions showed detection of RNase P whereas the undiluted sample did not show detection of RNase P. Additionally, primers and probes for COVID-19 nucleic acids added to the assay and N1 and N2 are not detected, but RNase P still shows amplification. A higher amplification in channel 4 calibrator due is observed due to detection of N3.

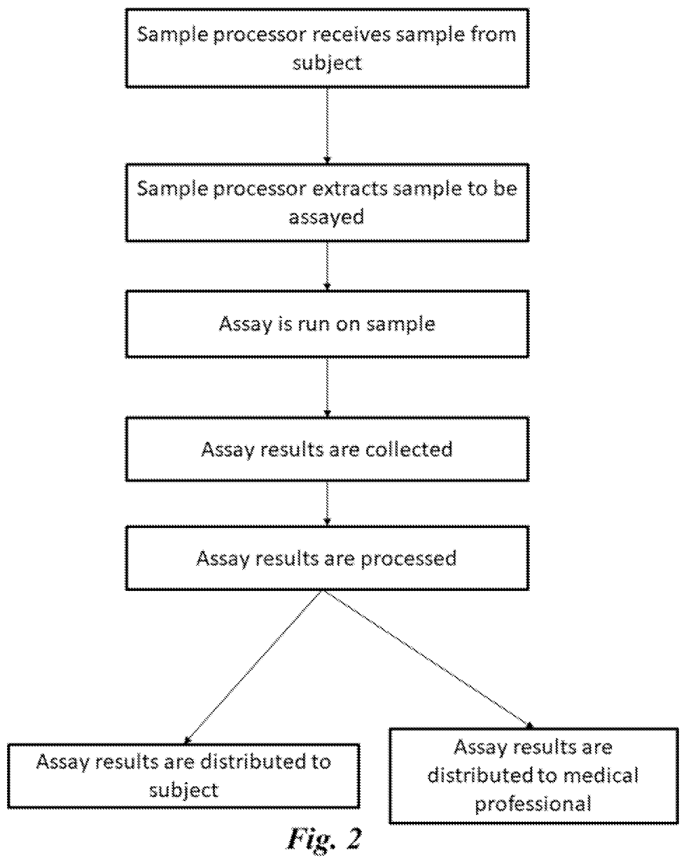

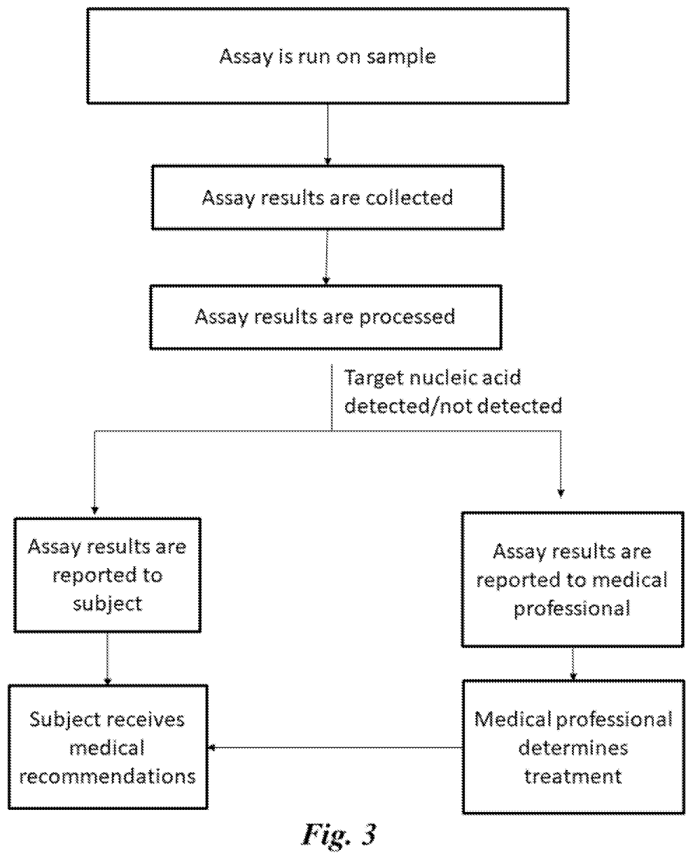

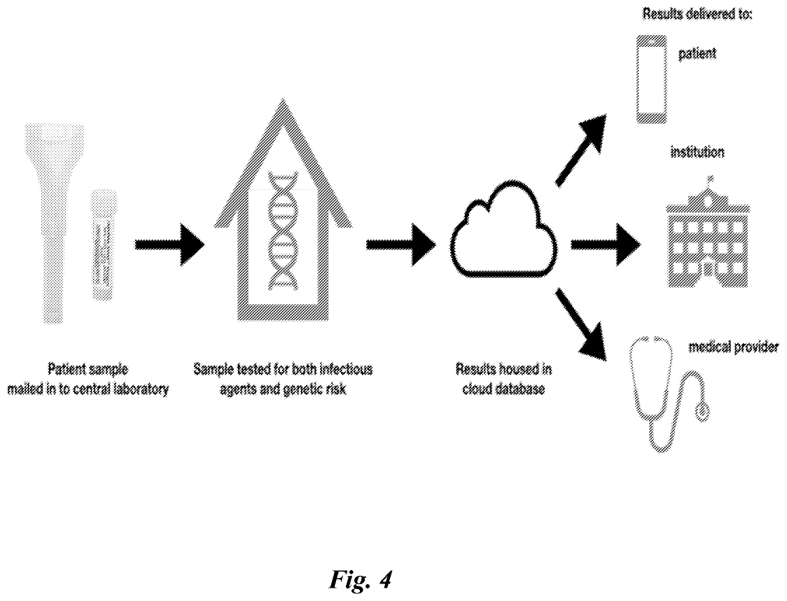

The results of the assay may then be uploaded into a remote server. For example, the results may be uploaded and sent to a subject's medical provider or an institution monitoring the spread of a disease. The results may also be sent to the subject directly. FIG. 2 and FIG. 3 show schematics of the assay as done by a testing facility or other individual(s) responsible for performing the assay, as was described in the Example. As shown in FIG. 3, the results may then be transmitted to another institution/or medical professional for monitoring or for providing recommendation for the subject. FIG. 4 shows a schematic of how the assay may be performed and how the data is generated. In additional to the data generated for detection of pathogens or an infectious disease, as demonstrated in the Example, the assay may also detect sequences in the person's genome that may indicate a genetic risk. These results can then be uploaded into a cloud database or other remote database for storage and transmission to or access by a variety or individuals and institutions which may use the results of the assay. The results may be obtained on a smart phone or other technological system which may display the results.

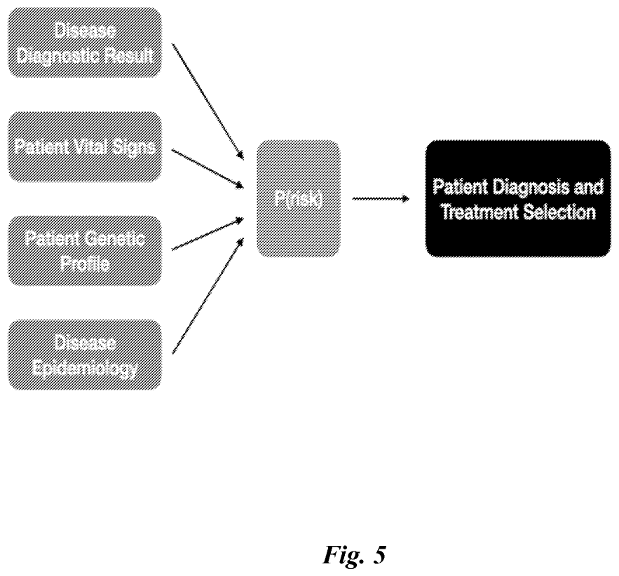

FIG. 5 shows a schematic of additional results or steps that may be used by the individual or other institutions for provide a diagnosis or treatment option. Using the assay results in conjunction with epidemiological information on the pathogen, as well as the subject's medical history, genetic profile, or vital signs, the results may include a risk score or probability score of the patient's disease severity or potential for complications or mortality risk. This may be outputted in the report and sent to the subject or institution to guide a diagnosis or treatment.

### Example 2: Multiplex Assay for Multiple Viral Pathogens

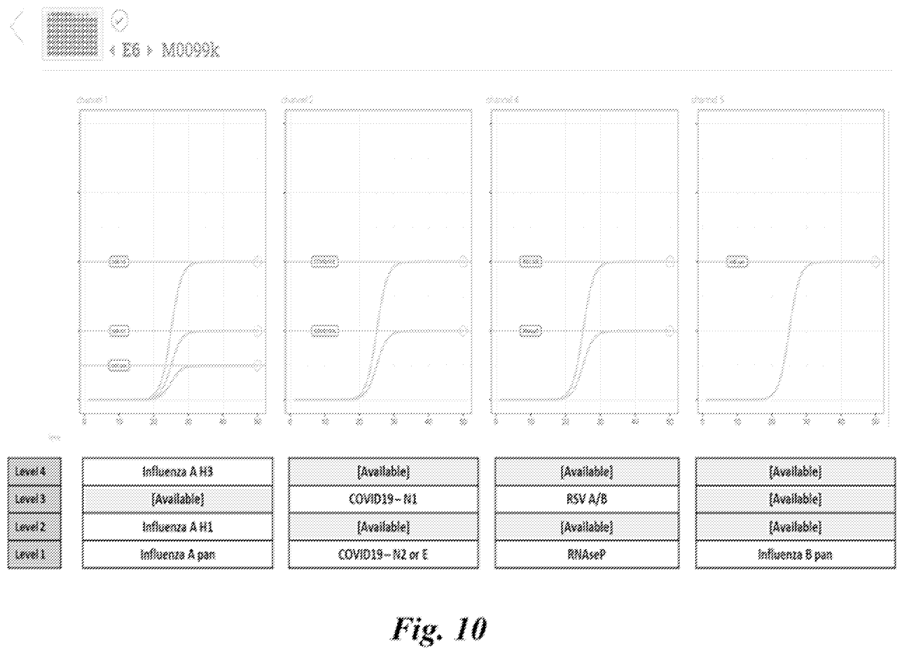

A sample is taken from an individual suspected of having COVID-19 is taken to be assayed. The symptoms of the individual are ambiguous and could be cause by multiple different pathogens and a determination of the pathogen is needed. An assay is performed on the sample such that multiple pathogens and genes can be detected in a single sample value. FIG. 10 demonstrates an assay set up for multiplexed detection. The detection is performed in 4 different channels (Channel 1-4) which each has a level of fluorescence that is correlated with a particular target nucleic acid. In Channel 1, the influenza A PB1 gene is identified as signal that plateaus at level 1, the influenza A H1 serotype is identified as signal that plateaus at level 2, the influenza A H3 serotype is identified as signal that plateaus at level 4. In Channel 1, the influenza A PB1 gene is identified as signal that plateaus at level 1, the influenza A H1 serotype is identified as signal that plateaus at level 2, the influenza A H3 serotype is identified as signal that plateaus at level 4.

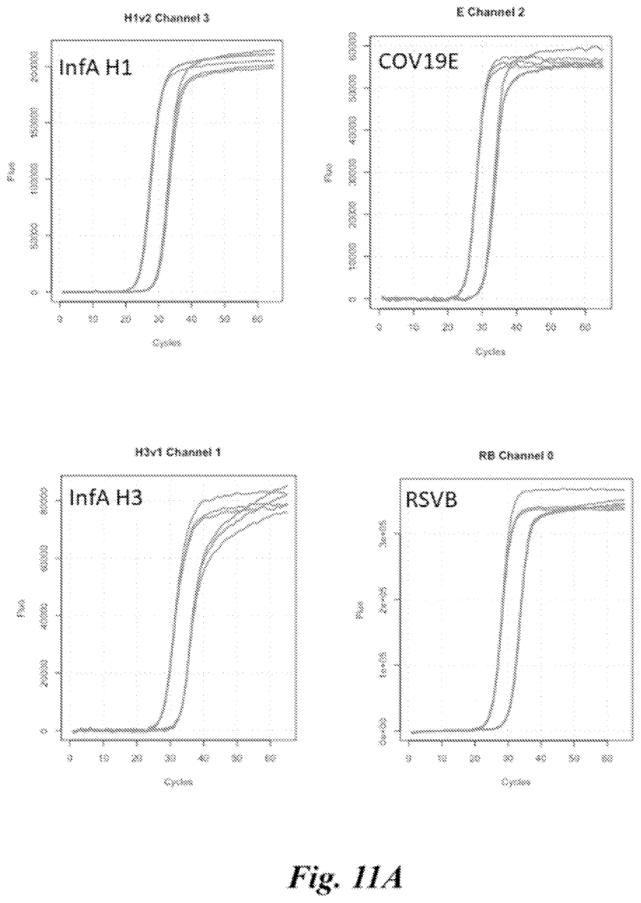

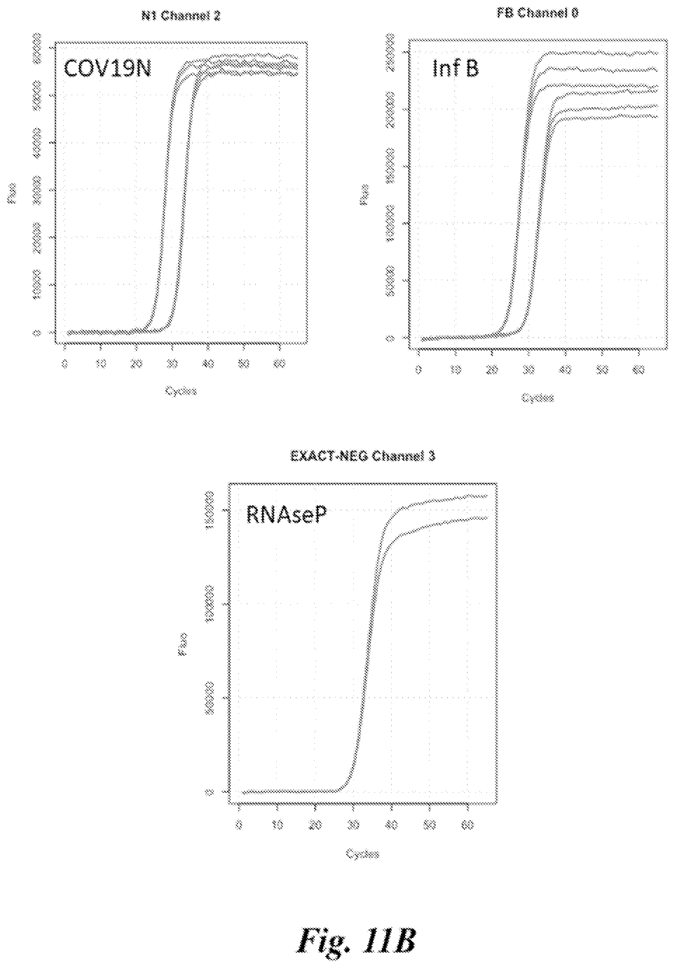

In Channel 2, the COVID 19 N2 or E gene is identified as signal that plateaus at level 1, the COVID 19 N1 is identified as signal that plateaus at level 3. In Channel 2, the COVID 19 N2 or E gene is identified as signal that plateaus at level 1, and the COVID 19 N1 is identified as signal that plateaus at level 3. In Channel 3, the RNase P gene is identified as signal that plateaus at level 1, and the RSV A/B gene is identified as signal that plateaus at level 3. In Channel 4, the influenza B NS gene is identified as signal that plateaus at level 1. The configuration of the assay may allow the multiplexing of up to 16 different target and can be configured in a way to make the determination of the presence of a particular pathogen easier. For example, as demonstrated, Channel 1 is used to detect influenza A genes, so a signal in channel 1, regardless of particular intensity or shape would indicate the presence of influenza A. A deconvolution of particular signals in channel 1 may then be performed to determine the serotype of influenza or a particular SNP present. Similar configurations may be designed, for example in channel 2 where the presence of a signal indicates the presence of COVID-19 or SARS-CoV-2 gene. FIG. 11A and B shows the output of the assay on the sample which has all the target nucleic acid. A signal is generated for all targets demonstrating that the assay can be performed in multiplex and provide a signal for each detected nucleic acid.

### Example 3: Detection of Viral RNA in Saliva Sample

A tube similar to one provided by Ancestry.com is used to receive a saliva sample. Sample is collected by filling the tube with saliva sample and up to a fill line (˜1.2 ml in volume). Matrix components and stabilizing components (e.g. Alcohol SDA 3C (ethanol denatured with 5% isopropanol), sodium acetate, and Tris) are allowed to mix with sample and the tube is sealed and inverted around 20 times to mix. The tube is subsequently incubated at room temperature for 30 mins. The sample can now be transferred to a testing location via mail service or other system of transport. The received sample is then vortexed for 10 secs. The contents of the tube are allowed to settle on the bottom of the tube or via the gentle tapping of the tube to move the contents to the bottom of the tube. Diluent of saliva matrix and 1×TE buffer (pH 8.0, 10 mM Tris, 0.1 mM EDTA) is made using a 1:5 matrix:TE ratio (400 μL of saliva matrix and 1600 μL TE). The diluent mixture is vortexed for 10 secs to mix. To generate a sample comprising a viral RNA of interest, 100 μL of saliva matrix, 300 μL of TE and 100 μL of Armored RNA Quant SARS-CoV-2 (105 copies/μL) is added to a fresh tube. Serial 10-fold dilutions of the RNA sample are generated by adding 20 μL of sample to 180 μL of previously made diluent in to Tube 2. Tube 2 is then vortexed and then spun down. 20 μL from Tube 2 is added to 180 μL of diluent in Tube 3. Serial dilution was repeated for additional tubes to generate a 1:10, 1:100, 1:1000, 1:10000, 1:100000 dilution.

75 μL of sample dilutions are transferred to a 96 well plate and covered with a foil heat sealer. The plate is heated using a heat sealer. The plate is spun down for 30 seconds and loaded into a thermocycler and allowed to incubate at 98° C. for 5 minutes and then is cooled to 4° C.

To run the PCR reaction, HDPCR mix containing primers and probes for SARS-CoV-2 N1, N2, N3 and RNase P, polymerase and reverse transcriptase enzymes, and NEB Luna Warmstart RT enzyme mix are added to a tube (1260 μL of HDPCR mix, 3150 μL of enzyme and 315 μL of Luna RT) to make a mastermix. The mastermix is vortexed for 10 seconds on a medium speed. Using an Eppendorf E3 repeater and 1 mL combitip 15 μL of mastermix is added to each well of three ABI Fast 96 well plates. Multiples plates are loaded with 6 replicates of the dilutions as well as 6 replicates of matrix sample containing no Armored RNA spike in. Additionally, 6 replicates of Exact Science Positive Control are loaded into each plate. To demonstrate consistency across platforms, plates were loaded in different thermocyclers. Plate 1 is loaded onto ABI QuantStudio 5 as shown and is run using the parameters below:

Plate 1 setup
Plate Column

| 1-4 | 5-8 | 9-12 |
|---|---|---|
| 50° C. | 55° C. | 60° C. |

Plate 1 Parameters

| Step | Cycles | Time | | Temperature ° C. | | |
|---|---|---|---|---|---|---|
| RT | 1 | 15 | minutes | 50 | 55 | 60 |
| (Veriflex) | | | | | | |

| | | | | |
|---|---|---|---|---|
| Activation | 1 | 2 | minutes | 95 |
| Cycling | 65 | 3 | seconds | 3 |
| | | 30 | seconds | 58 |

Plate 2 is loaded onto ABI QuantStudio 7 and run as below:

Plate 2 Parameters

| Step | Cycles | Time | | Temperature ° C. |
|---|---|---|---|---|
| RT | 1 | 15 | minutes | 50 |
| Activation | 1 | 2 | minutes | 95 |
| Cycling | 65 | 3 | seconds | 3 |
| | | 30 | seconds | 58 |

Plate 3 is loaded onto Viia 7 and run as shown below

Plate 3 Parameters

| Step | Cycles | Time | | Temperature ° C. |
|---|---|---|---|---|
| RT | 1 | 15 | minutes | 50 |
| Activation | 1 | 2 | minutes | 95 |
| Cycling | 65 | 3 | seconds | 3 |
| | | 30 | seconds | 58 |

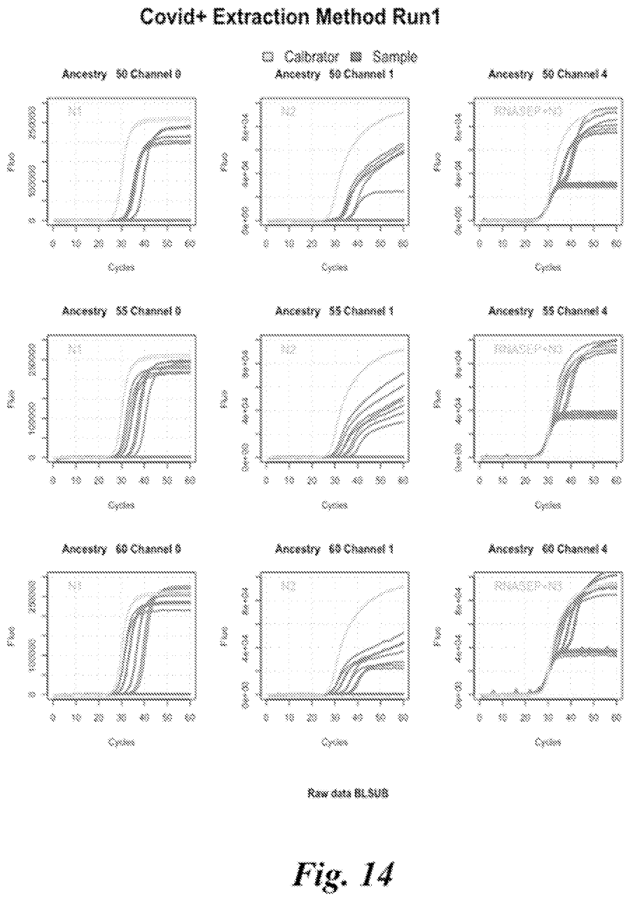

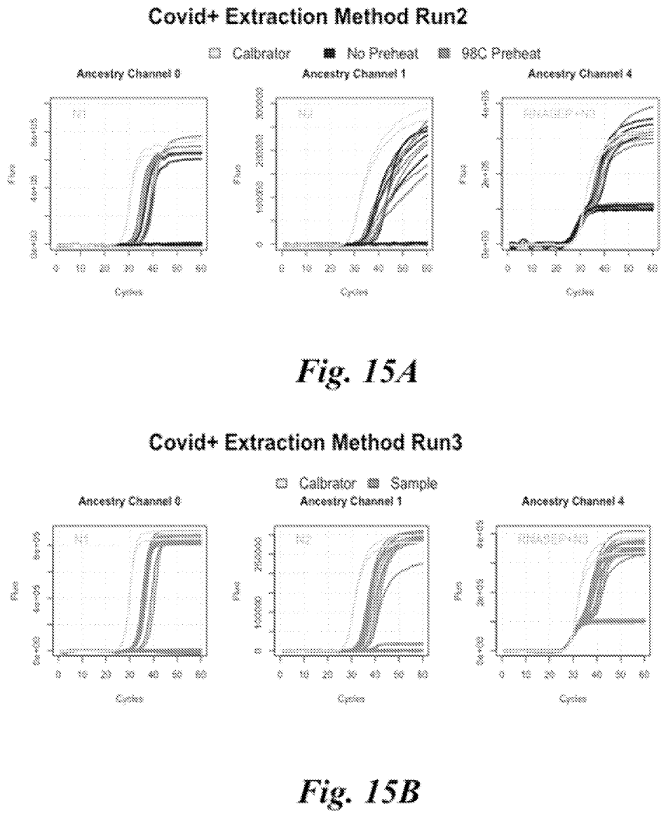

Shown in FIG. 14, signals are observed for multiple dilutions ran on Plate 1. In the 55° C. and 60° C. reactions, separation of signal curves for each dilution was observed. Distinct signals are observed in Channel 4 for RNase P and N3 allowing observation of both in the sample. As shown in FIG. 15A, signal was observed for preheated and non-preheated samples run in Plate 2, with improved signal in the pre-heated sample. As shown in FIG. 15B signals are observed for the dilutions run on Plate 3. N2 showed improved signal generation compared to samples run on Plate 1. Additionally, a subsequent run was performed with modified conditions are used in the PCR, using the higher reverse transcriptase temperatures of plate 1 (65° C.) and the increased annealing time of 60 secs used in run 3 with improved signal generation.

A fourth plate is set up using similar dilution conditions and also testing dilutions in TE buffer alone without addition of the matrix in the diluent. The parameters for plate 4 are shown below:

Plate 4 Parameters

| Step | Cycles | Time | | Temperature ° C. | | |
|---|---|---|---|---|---|---|
| RT | 1 | 15 | minutes | 55 | 57.5 | 60 |
| (Veriflex) | | | | | | |

| | | | | |
|---|---|---|---|---|
| Activation | 1 | 2 | minutes | 95 |
| Cycling | 65 | 3 | seconds | 3 |
| | | 30 | seconds | 58 |

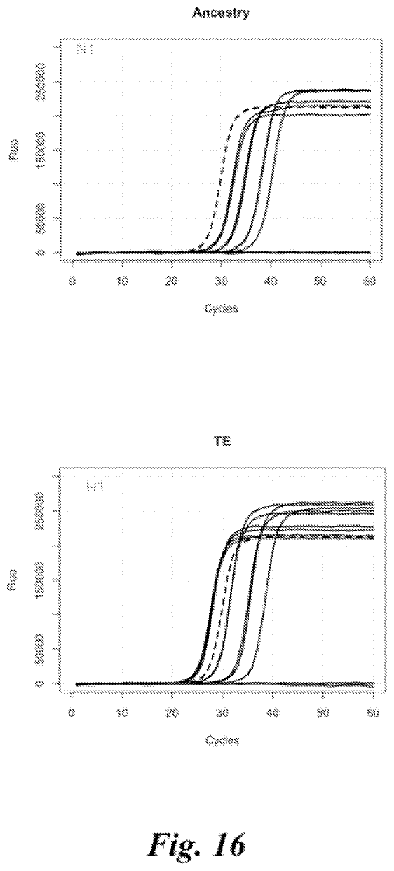

Results of the run for detection of N1 are shown in the FIG. 16 and compare the signal of the sample diluted with matrix versus those diluted in TE buffer alone. Signals are observed for multiple dilutions in both TE and Matrix diluents. The detection of N1is shown in the below table indicating the number of duplicates run and those with successful detection at a copy number of viral RNA. At 105 copies, RNA is detected at all conditions, with the detection becoming increasingly more difficult at lower copy numbers. The saliva matrix may have some minor inhibitory properties on the PCR reaction but may also have benefits in maintaining sample integrity prior to testing.

Accuracy and limits of detection results for runs

| | Run 1 Saliva matrix | | | | | | | | |
|---|---|---|---|---|---|---|---|---|---|
| Copy per | RT hold | | | Run 4 Saliva matrix | | | Run 4 TE | | |
| Reaction | 50° C. | 55° C. | 60° C. | 55° C. | 57.5° C. | 60° C. | 55° C. | 57.5° C. | 60° C. |
| 10⁵ | 2/2 | 2/2 | 2/2 | 2/2 | 2/2 | 2/2 | 2/2 | 2/2 | 2/2 |
| 10⁴ | 2/2 | 2/2 | 2/2 | 2/2 | 2/2 | 2/2 | 2/2 | 2/2 | 2/2 |
| 10³ | 2/2 | 2/2 | 2/2 | 2/2 | 2/2 | 2/2 | 2/2 | 2/2 | 2/2 |
| 10² | 0/2 | 1/2 | 2/2 | 1/2 | 0/2 | 0/2 | 2/2 | 2/2 | 2/2 |
| 10 | 0/2 | 0/2 | 0/2 | 0/2 | 0/2 | 0/2 | 0/2 | 1/2 | 1/2 |
| 1 | 0/2 | 0/2 | 0/2 | 0/2 | 0/2 | 0/2 | 0/2 | 0/2 | 0/2 |

### Example 4: Primer Design

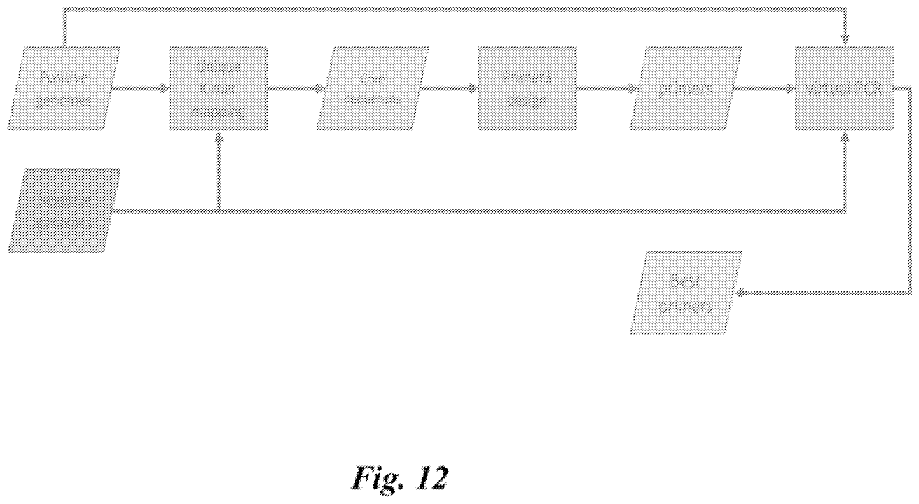

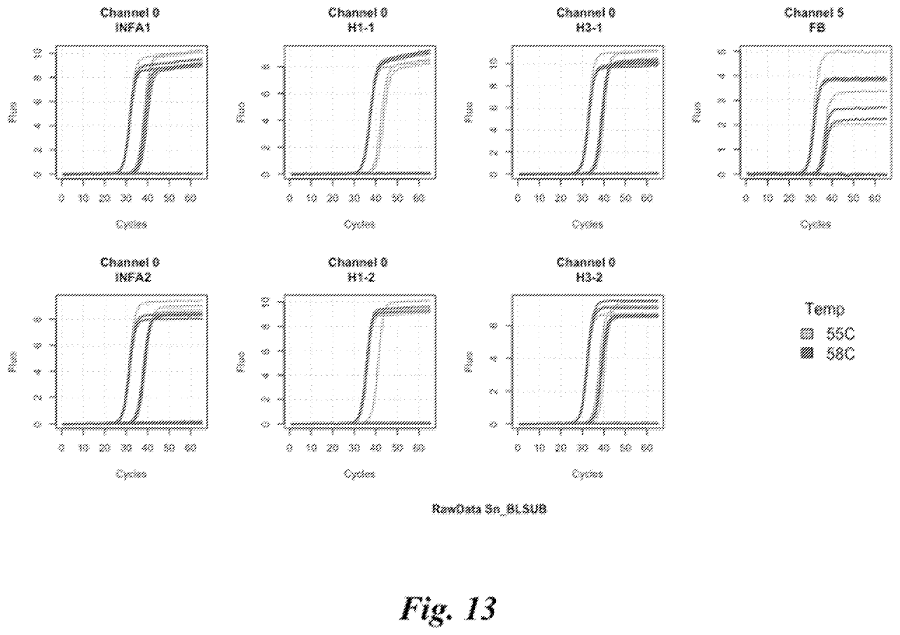

Primers for the assays shown in Examples 1-3 can be designed to optimize the amount of signal and prevent false negatives or positives. In multiplexed assays, the primers for different targets may interact unfavorably or a primer may be prone to interact with an off-target. In multiplexed assays, the off-target may be a target of another primer in the multiplexed assay, creating a false positive signal. FIG. 12 shows a schematic for designing the primers. The positive genome (e.g. a genome that has a target nucleic acid sequence) and a negative genome (e.g. a genome that the primer should not react with) are inputted a program and unique k-mer (oligonucleotides of k length) are determined that are in the positive genome but not in the negative genome. A core sequence of the multiple unique k-mer sequences are identified as a sequence that would be useful for designing primers to. Using a software suite like Primer3, or other database that can optimize or design primers to a particular sequence based on different particular parameters such as melting temperature, product size, GC content, primer dimers, probability of amplification of a wrong sequence, the primers to the core sequence can be designed. A virtual PCR can then be performed to identify the best primers or primers that have desired quality. This method can be optimized for different multiplexed assays depending on the multiple targets. In the case of targets disclosed in Example 2, sequences from NCBI and GISAID for Influenza A (PB1 H1, H3), Influenza B (NS), RSVA, RSVB, and COVID are used as the positive sequences. Negative sequences include sequences to SARS, MERS and other sequences found in other respiratory viruses. FIG. 13 shows graphs generated from a singleplex assays showing that the primers are able to amplify the respective target at both 55° C. and 58° C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1.-78. (canceled)

79. A method of identifying a subject as having a disease associated with an infectious pathogen, the method comprising:
(a) receiving from a subject a saliva sample collection tube comprising a saliva sample derived from said subject;
(b) combining at least a subset of said saliva sample with nucleic acid detection assay reagents;
(c) performing a nucleic acid detection assay reaction on said saliva sample, wherein said nucleic acid detection assay generates a signal in the presence of one or more target nucleic acids associated with said infectious pathogen; and
(d) detecting a presence or absence of said signal of said nucleic acid detection assay reaction to detect the presence or absence of said one or more target nucleic acids.

80. The method of claim 79, wherein no chemical reagents are added to the received saliva sample between steps a) and b).

81. The method of claim 79, wherein said received saliva sample is not chemically processed prior to addition of said nucleic detection assay reagents.

82. The method of claim 79, wherein said saliva sample is heated between steps a) and b)

83. The method of claim 79, wherein said saliva sample is not subjected to an amplification reaction prior to performing said assay reaction.

84. The method of claim 79, wherein said saliva sample is diluted prior to performing said assay.

85. The method of claim 79, wherein nucleic acids from said saliva sample are not subjected to an extraction or purification reaction between steps a) and b).

86. The method of claim 79, wherein said one or more target nucleic acids comprises a single nucleotide polymorphism (SNP).

87. The method of claim 79, wherein said one or more target nucleic acids comprises a genetic marker in said subject associated with a predisposition of a disease linked to said infectious pathogen.

88. The method of claim 87, further comprising, using said presence or absence of said target nucleic acids to generate a genotype for said subject.

89. The method of claim 88, further comprising using said genotype and said presence of said infectious disease agent to generate a risk metric for said subject.

90. The method of claim 88, wherein said genotype comprises sequences related to immune response.

91. The method of claim 90, wherein sequences related to immune response comprise sequences associated with increased immunity or decreased immunity to said infectious pathogen.

92. The method of claim 79, wherein said infectious pathogen is a bacteria, virus or fungus.

93. The method of claim 92, wherein said virus is an influenza virus, hepatitis virus, herpesvirus, papillomavirus, or coronavirus.

94. The method of claim 79, wherein said target nucleic acids comprise sequences selected from members of the group consisting of COVID N gene, COVID E gene, influenza A PB1 gene, influenza HA gene, influenza B NS gene, and Respiratory syncytial virus A/B F gene.

95. The method of claim 94, wherein said influenza HA gene comprises a sequence of a H1 or a H3 serotype.

96. The method of claim 95, wherein said detecting is performed in a single channel.

97. The method of claim 96, wherein said detecting is performed using a multiple channel instrument.

98. The method of claim 79, further comprises quantifying the one or more target nucleic acids associated with said infectious pathogen.


SEQUENCE LISTING (36 sequences)

SEQ ID NO: 1 - DNA, 20 bp, Artificial Sequence
  Description of Artificial Sequence: Synthetic nucleotide sequence
gaccccaaaatcagcgaaat

SEQ ID NO: 2 - DNA, 24 bp, Artificial Sequence
  Description of Artificial Sequence: Synthetic nucleotide sequence
tctggttactgccagttgaatctg

SEQ ID NO: 3 - DNA, 20 bp, Artificial Sequence
  Description of Artificial Sequence: Synthetic nucleotide sequence
ttacaaacattggccgcaaa

SEQ ID NO: 4 - DNA, 18 bp, Artificial Sequence
  Description of Artificial Sequence: Synthetic nucleotide sequence
gcgcgacattccgaagaa

SEQ ID NO: 5 - DNA, 19 bp, Artificial Sequence
  Description of Artificial Sequence: Synthetic nucleotide sequence
agatttggacctgcgagcg

SEQ ID NO: 6 - DNA, 20 bp, Artificial Sequence
  Description of Artificial Sequence: Synthetic nucleotide sequence
gagcggctgtctccacaagt

SEQ ID NO: 7 - DNA, 24 bp, Artificial Sequence
  Description of Artificial Sequence: Synthetic nucleotide sequence
ctcagacgattttgccctcatagt

SEQ ID NO: 8 - DNA, 27 bp, Artificial Sequence
  Description of Artificial Sequence: Synthetic nucleotide sequence
gaatacaagcaggagtggatagattct

SEQ ID NO: 9 - DNA, 27 bp, Artificial Sequence
  Description of Artificial Sequence: Synthetic nucleotide sequence
cactaacttgcaggtcctatagaatct

SEQ ID NO: 10 - DNA, 25 bp, Artificial Sequence
  Description of Artificial Sequence: Synthetic nucleotide sequence
gctagtgaattcaaatgtccctgtt

SEQ ID NO: 11 - DNA, 24 bp, Artificial Sequence
  Description of Artificial Sequence: Synthetic nucleotide sequence
cggctgctttgaattttaccacaa

SEQ ID NO: 12 - DNA, 23 bp, Artificial Sequence
  Description of Artificial Sequence: Synthetic nucleotide sequence
gttgatgatggtttcctggacat

SEQ ID NO: 13 - DNA, 25 bp, Artificial Sequence
  Description of Artificial Sequence: Synthetic nucleotide sequence
gcttcctctgagtattttgggtagt

SEQ ID NO: 14 - DNA, 25 bp, Artificial Sequence
  Description of Artificial Sequence: Synthetic nucleotide sequence
cgtgatagtccaaagttctttcgtt

SEQ ID NO: 15 - DNA, 22 bp, Artificial Sequence
  Description of Artificial Sequence: Synthetic nucleotide sequence
ggaagaggacaagcagcagatc

SEQ ID NO: 16 - DNA, 21 bp, Artificial Sequence
  Description of Artificial Sequence: Synthetic nucleotide sequence
gcttcttgttgccctggagaa

SEQ ID NO: 17 - DNA, 25 bp, Artificial Sequence
  Description of Artificial Sequence: Synthetic nucleotide sequence
caatctgatggaatttctcgttggt

SEQ ID NO: 18 - DNA, 21 bp, Artificial Sequence
  Description of Artificial Sequence: Synthetic nucleotide sequence
tgcaggcattgtcacatttgt

SEQ ID NO: 19 - DNA, 26 bp, Artificial Sequence
  Description of Artificial Sequence: Synthetic nucleotide sequence
gatgatcttacagtggaggatgaaga

SEQ ID NO: 20 - DNA, 24 bp, Artificial Sequence
  Description of Artificial Sequence: Synthetic nucleotide sequence
cttctggtgataatcggtgctctt

SEQ ID NO: 21 - DNA, 26 bp, Artificial Sequence
  Description of Artificial Sequence: Synthetic nucleotide sequence
tggtactgtgacaatgcaggatcagt

SEQ ID NO: 22 - skipped

SEQ ID NO: 23 - DNA, 29 bp, Artificial Sequence
  Description of Artificial Sequence: Synthetic nucleotide sequence
gcatatgttgtacagctacctatctatgg

SEQ ID NO: 24 - DNA, 27 bp, Artificial Sequence
  Description of Artificial Sequence: Synthetic nucleotide sequence
ccatcctctatcagtccttgttaaaca

SEQ ID NO: 25 - DNA, 24 bp, Artificial Sequence
  Description of Artificial Sequence: Synthetic nucleotide sequence
accccgcattacgtttggtggacc

SEQ ID NO: 26 - DNA, 23 bp, Artificial Sequence
  Description of Artificial Sequence: Synthetic nucleotide sequence
acaatttgcccccagcgcttcag

SEQ ID NO: 27 - DNA, 23 bp, Artificial Sequence
  Description of Artificial Sequence: Synthetic nucleotide sequence
ttctgacctgaaggctctgcgcg

SEQ ID NO: 28 - DNA, 24 bp, Artificial Sequence
  Description of Artificial Sequence: Synthetic nucleotide sequence
tgagggaatacaagcaggagtgga

SEQ ID NO: 29 - DNA, 25 bp, Artificial Sequence
  Description of Artificial Sequence: Synthetic nucleotide sequence
aggacctgcaagttagtgggaatca

SEQ ID NO: 30 - DNA, 25 bp, Artificial Sequence
  Description of Artificial Sequence: Synthetic nucleotide sequence
tgcatggaaagtgtcaagaatggga

SEQ ID NO: 31 - DNA, 26 bp, Artificial Sequence
  Description of Artificial Sequence: Synthetic nucleotide sequence
tggacttacaatgccgaactgttggt

SEQ ID NO: 32 - DNA, 25 bp, Artificial Sequence
  Description of Artificial Sequence: Synthetic nucleotide sequence
agcactcaagcagcaatcgatcaaa

SEQ ID NO: 33 - DNA, 25 bp, Artificial Sequence
  Description of Artificial Sequence: Synthetic nucleotide sequence
agcaactgagggaaaatgctgagga

SEQ ID NO: 34 - DNA, 27 bp, Artificial Sequence
  Description of Artificial Sequence: Synthetic nucleotide sequence
aggacattcaaagccaattcgagcagc

SEQ ID NO: 35 - DNA, 23 bp, Artificial Sequence
  Description of Artificial Sequence: Synthetic nucleotide sequence
gttaggtgttggatctgcaatcg

SEQ ID NO: 36 - DNA, 24 bp, Artificial Sequence
  Description of Artificial Sequence: Synthetic nucleotide sequence
cagccttgtttgtggatagtagag